(19) United States
(12) Patent Application Publication
(10) Pub. No.: US 2025/0082728 A1
(43) Pub. Date: Mar. 13, 2025

(54) INTERLEUKIN 4 AND/OR INTERLEUKIN 13-BASED EYE COMPOSITIONS

(71) Applicant: UNIVERSITY OF PITTSBURGH - OF THE COMMONWEALTH SYSTEM OF HIGHER EDUCATION, Pittsburgh, PA (US)

(72) Inventors: Alexis Lauren NOLFI, Moon Township, PA (US); Bryan Nicklaus BROWN, Pittsburgh, PA (US); Mangesh KULKARNI, Sewickley, PA (US); Vishal JHANJI, Pittsburgh, PA (US)

(73) Assignee: UNIVERSITY OF PITTSBURGH - OF THE COMMONWEALTH SYSTEM OF HIGHER EDUCATION, Pittsburgh, PA (US)

(21) Appl. No.: 18/956,193

(22) Filed: Nov. 22, 2024

Related U.S. Application Data
(63) Continuation of application No. PCT/US2023/023408, filed on May 24, 2023.
(60) Provisional application No. 63/345,301, filed on May 24, 2022.

(51) Int. Cl. A61K 38/20; A61K 9/00; A61K 31/726; A61K 47/64; A61P 27/02

(52) U.S. Cl. CPC A61K 38/2026; A61K 9/0048; A61K 31/726; A61K 38/2086; A61K 47/643; A61P 27/02

(57) ABSTRACT

Described here are ophthalmic compositions and methods for treatment of dry eye disease. One ophthalmic composition includes an effective amount of interleukin-4 (IL-4) in a free uncomplexed form, or as a complex with a dermatan sulfate. A second ophthalmic composition includes an effective amount of interleukin-13 (IL-13) in a free uncomplexed form, or as a complex with a dermatan sulfate. A third ophthalmic composition includes effective amounts of IL-4 and IL-13 in a free uncomplexed form, or as a complex with a dermatan sulfate. A fourth ophthalmic composition includes at least one activator of interleukin-4 receptor signaling in a free uncomplexed form, or as a complex with a dermatan sulfate.

### CROSS-REFERENCE TO RELATED APPLICATIONS

This application is a continuation of International Application No. PCT/US2023/023408 filed on May 24, 2023, which claims priority to U.S. Provisional Patent Application Ser. No. 63/345,301 filed on May 24, 2022, both of which are hereby incorporated by reference in their entireties.

### FIELD OF THE INVENTION

The presently disclosed subject matter relates to cytokine compositions and methods of delivering such for treatment of ocular disorders.

### BACKGROUND OF THE INVENTION

Dry Eye Disease (DED) is among the most frequent ocular morbidities, estimated to affect 7.4 to 33.7% of the population worldwide (1). Severe, chronic DED can lead to vision-threatening corneal ulceration, perforation, scarring, negative psychological consequences, and reduced quality of life for patients. Patients frequently complain of itchiness, blurred vision, and ongoing pain-sometimes so severe that they report disruptions in sleep (2). Tasks that require long periods of visual concentration such as reading a book, working at a computer, or operating a vehicle, can also become difficult or impossible and can lead to feelings of loss of autonomy and independence (3). Additionally, DED sufferers often have a poor self-image due to the appearance of perpetually red, irritated eyes and often suffer anxiety and depression characteristic of chronic illnesses (4). The need for frequent physician visits, medications, and procedures, as well as loss of productivity due to days of missed work are financially burdensome on multiple levels. While the direct and indirect costs of DED are proportional to the severity of the disease, the average cost per patient in the United States was estimated in 2008 to be over $11,000, which equates to total societal costs in excess of $50 billion annually (5).

In healthy eyes, the tear film is made up of an oily outer layer, an aqueous middle layer and a mucous (mucin) inner layer. Proper tear film formation is crucial for proper eye function, and depends on interactions between tear-producing glands, epithelium, mucin producing goblet cells, immune cells (e.g., NK, dendritic, macrophage, T cells), and endogenous biomolecules (6). DED is recognized to be a multifactorial disorder that encompasses a self-perpetuating cycle of tear film disruption, tear hyperosmolarity, surface desiccation, ocular barrier disruption, and inflammation (7). Each of these individual components affects the others and can also be modulated by patient-specific extrinsic factors (e.g., environmental, contact lens usage, medication, surgical interventions) and intrinsic factors (e.g., sex, age, autoimmune disorders) (8). For these reasons, it is difficult to define and treat a single cause of DED in an individual patient and, irrespective of the initiating insult, the result is almost always an unresolved, self-perpetuating cycle of tear film instability, tissue damage and inflammation (1).

Current treatment options are largely limited to either symptomatic over-the-counter relief (e.g., artificial tears) or prescription medications targeting the adaptive immune system. There are a myriad of artificial tear formulations comprising demulcents, emollients, preservatives, and buffers that mimic the composition, pH, and osmolarity of natural tears. The goal of such formulations is to provide lubrication, stabilize the tear film, and retard desiccation, thereby mitigating DED symptoms. Artificial tears however do little to address the underlying cellular and inflammatory mechanisms of DED, nor do they offer long-term relief. Prescription medications include topical cyclosporine (e.g., Restasis®, Cequa™, Klarity-C/CL Drops®) or topical lifitegrast (Xiidra®). Cyclosporine and lifitegrast broadly target T cell-mediated pathways of inflammation by reducing migration, proliferation, activation, and inflammatory mediator secretion (9, 10). However, these treatments are associated with side effects including, burning, irritation, and blurry vision. They also require frequent instillation of drops, which in conjunction with the above-described side effects, can result in patients not complying with the prescribed treatment (11). Furthermore, bolus delivery and product loss are major limitations of eye drops, as the therapeutic agent is only in contact with the ocular surface for a short period, limiting efficacy (30).

As a result of these limitations, DED continues to progress, warranting additional surgical, systemic, and/or steroidal interventions, leading to undesirable risk profiles. For example, steroid eye drops are occasionally used; however, the side effects (e.g., cataract development, glaucoma) can be debilitating (12). Oral treatments, such as doxycycline, have their own set of concerns, coupled with the limitation of using a systemic drug to treat a localized condition (13). Punctal plugs are an option for some, with limitations ranging from easy dislodgement to irritation of the eye and ducts (14). When these fail to adequately control symptoms, more obscure treatments are considered including, permanent surgical punctal cauterization, long-term use of topical steroids, insertion of rigid scleral contact lenses, and use of autologous serum eye drops, among others (12). These treatments are however fraught with risks that outweigh their potential benefits. There is hence a need in the art for new compositions and methods that provide improved treatments for DED.

### SUMMARY OF THE INVENTION

The present invention is directed to ophthalmic compositions and methods for treating ocular disorders. The disclosed invention is based, in part, on the discovery that the ophthalmic compositions improves tear film stability and reduce inflammation, thereby treating ocular disorders.

The present invention is directed to a composition for treating an ocular disorder comprising an effective amount of interleukin-4 (IL-4) and/or interleukin-13 (IL-13). The present invention is also directed to a composition for treating an ocular disorder comprising an effective amount of IL-4 and/or IL-13, complexed with a dermatan sulfate. The present invention is further directed to a composition comprising at least one activator of interleukin-4 receptor (IL-4R) signaling. In non-limiting embodiments, the at least one activator of IL-4R signaling can comprise IL-4, IL-13, or a combination of IL-4 and IL-13.

In non-limiting embodiments, the IL-4 and/or IL-13 can be complexed with the dermatan sulfate in a ratio from about 1:1000 (weight/weight, w/w) to about 1:120000 (w/w). In certain embodiments, the ratio can be about 1:60000 (w/w).

In certain embodiments, the composition further includes a buffer with a carrier protein. In non-limiting embodiments, the buffer can include phosphate-buffered saline (PBS), and the carrier protein can include bovine serum albumin (BSA).

In certain embodiments, the composition can comprise IL-4 and/or IL-13, each at a concentration range from about 0.1 ng to about 50 ng. In non-limiting embodiments, a concentration of the dermatan sulfate can range from about 100 ng to about 6000 μg.

In certain embodiments, the composition can be in a form of eye drop solution, suspensions, ointments, or sprays. In non-limiting embodiments, a concentration of the IL-4 and/or IL-13 can be from about 1 ng of the IL-4 per 5 μL of the solution to about 1 ng of the IL-4 per 500 μL of the solution and/or, from about 1 ng of the IL-13 per 5 μL of the solution to about 1 ng of the IL-13 per 500 μL of the solution.

The present invention is directed to a method for treating ocular disorders comprising administering a composition comprising an effective amount of interleukin-4 (IL-4) and/or IL-13 to an eye of a subject. The present invention is also directed to a composition for treating an ocular disorder comprising administering a composition comprising an effective amount of IL-4 and/or IL-13 complexed with a dermatan sulfate to an eye of a subject. The present invention is also directed to a composition for treating an ocular disorder comprising administering a composition comprising at least one activator of IL-4R signaling to an eye of a subject.

In certain embodiments, the composition can be administered topically to the eye of the subject. In non-limiting embodiments, the composition can be administered to the eye of the subject in a dose range from about 0.1 ng per eye to about 10 ng per eye.

In certain embodiments, the composition is administered in a form of an eye drop solution. In non-limiting embodiments, the dosage for the eye can be about 2 drops of the solution, and each drop can include about 30 μL to about 100 μL of the eye drop solution. In non-limiting embodiments, each drop of the eye drop solution can correspond to about 0.1 ng to about 50 ng of IL-4 and/or about 0.1 ng to about 50 ng of IL-13. In non-limiting embodiments, the composition can be administered daily or twice daily to the eye of the subject for about 14 days.

In certain embodiments, the ratio between the IL-4 and the dermatan sulfate can be about 1:60000 (w/w) and the ratio between the IL-4 and the dermatan sulfate can be about 1:60000 (w/w). In non-limiting embodiments, a concentration of the IL-4 can range from about 0.1 ng to about 50 ng. In non-limiting embodiments, a concentration of the IL-13 can range from about 0.1 ng to about 50 ng. In non-limiting embodiments, a concentration of the dermatan sulfate can range from about 100 ng to about 6000 μg.

In certain embodiments, the ocular disorder can include conjunctivitis, dry eye syndrome, hematomas, corneal abrasion, or combinations thereof. In non-limiting embodiments, the ocular disorder is dry eye disease.

### BRIEF DESCRIPTION OF THE FIGURES

FIGS. 1A-1D illustrate an exemplary method of producing a surgical model of DED in rabbits. FIG. 1A illustrates the first step in isolation of the inferior lacrimal gland. FIG. 1B illustrates removal of the inferior lacrimal gland. FIG. 1C illustrates the first step in isolation of the nictitating membrane. FIG. 1D illustrates removal of the nictitating membrane.

FIGS. 2A-2E provide exemplary assessment of corneal damage using fluorescein. FIG. 2A shows a representative fluorescein staining image of control rabbits (“baseline”, did not undergo surgery) that have not undergone surgical removal of the inferior lacrimal gland and nictitating membrane. FIG. 2B shows representative fluorescein staining images of animals in the PBS or IL-4 treatment groups at initiation of treatment. FIG. 2C shows representative fluorescein staining images of animals in the PBS and IL-4 treatment groups at day 7 after start of treatment. FIG. 2D shows representative fluorescein staining images of animals in the PBS and IL-4 treatment groups at day 14 after start of treatment. FIG. 2E provides quantification of the percent area of corneal damage in the indicated treatment groups.

FIGS. 3A-3J provide exemplary analysis of the effects of IL-4 on goblet cells macrophages and T cells in ocular sections. FIG. 3A shows representative Periodic Acid Schiff (PAS) staining of goblet cells in the ocular sections of control animals that have not undergone surgery. FIG. 3B shows representative PAS staining of goblet cells in animals having DED following PBS treatment. FIG. 3C shows representative PAS staining of goblet cells in rabbits having DED following IL-4 treatment. FIG. 3D provides quantitative assessment of the number of goblet cells in the indicated treatment groups. FIG. 3E shows representative RAM11 antibody staining for macrophages in healthy animals that have not undergone surgery. FIG. 3F shows representative RAM11 antibody staining in rabbits having DED following PBS treatment. FIG. 3G shows representative RAM11 antibody staining in rabbits having DED following IL-4 treatment. FIG. 3H shows representative CD3 staining for T cells in healthy animals that have not undergone surgery. FIG. 3I shows representative CD3 antibody staining in rabbits having DED following PBS treatment. FIG. 3J shows representative CD3 staining in rabbits having DED following IL-4 treatment.

FIGS. 4A-4E provide exemplary analysis of the effects of IL-4 on macrophage polarization. FIG. 4A shows representative immunocytochemistry of Arg-1 staining in naive macrophages (untreated, resting macrophages). FIG. 4B shows Arg-1 staining in macrophages incubated with DS. FIG. 4C shows Arg-1 staining in macrophages incubated with DS/IL-4 complexes (2 ng IL-4). FIG. 4D shows Arg-1 staining in macrophages incubated with uncomplexed IL-4 (20 ng IL-4). FIG. 4E provides quantification of Arg-1 expression in the indicated treatment groups.

### DETAILED DESCRIPTION OF THE INVENTION

The presently disclosed subject matter provides ophthalmic compositions that can simultaneously address both tear film instability and inflammation for treating ocular disorders. In certain embodiments, an exemplary composition can include an effective amount of interleukin-4 (IL-4) and a dermatan sulfate. The presently disclosed subject matter further provides single-cytokine drug delivery methods for treating ocular disorders using the ophthalmic composition.

For clarity of description, and not by way of limitation, the detailed description of the invention is divided into the following subsections:
1. Definitions;
2. Ophthalmic composition;
3. Methods of manufacturing the ophthalmic composition; and
4. Methods of treating ocular disorders.

### 1. Definitions

As used herein, the following terms have the meanings ascribed to them below, unless specified otherwise. Abbreviations used herein have their conventional meaning within the chemical and biological arts.

As used in the specification and the appended claims, the singular forms “a,” “an,” and “the” include plural referents unless the context clearly dictates otherwise. Thus, for example, a reference to “a compound” includes mixtures of compounds.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The term “active agent” refers to an agent that is capable of having a physiological effect when administered to a subject. In certain embodiments, the term “active agent” refers to an agent that can improve the tear film instability and inflammation of a subject, including, for example, but not limited to, cytokines (e.g., IL-4).

As used herein, the term “activator” or “signaling activator” refers to agents or compounds that activate a referenced signaling pathway. For example, but not by way of limitation, an “activator of interleukin-4 receptor signaling” activates signaling pathways associated with the interleukin-4 receptor.

As used herein, the term “administering” can mean any suitable route, e.g., via topical administration (e.g., eye drops, suspensions, ointments, spray), intraocular, intravitreal, intracameral, subconjunctival, sub-Tenon, or retrobulbar administration.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of,” and “consisting essentially of,” the embodiments or elements presented herein, whether explicitly set forth or not.

The term “dosage” is intended to encompass a formulation expressed in terms of total amounts for a given timeframe, for example, as μg/kg/hr, μg/kg/day, mg/kg/day, or mg/kg/hr. The dosage is the amount of an ingredient administered in accordance with a particular dosage regimen. A “dose” is an amount of an agent administered to a mammal in a unit volume or mass, e.g., an absolute unit dose expressed in mg of the agent. The dose depends on the concentration of the agent in the formulation, e.g., in moles per liter (M), mass per volume (m/v), or mass per mass (m/m). The two terms are closely related, as a particular dosage results from the regimen of administration of a dose or doses of the formulation. The particular meaning, in any case, will be apparent from the context.

The term “effective amount,” as used herein, refers to the amount of active agent sufficient to treat, prevent, or manage a disease. Further, a therapeutically effective amount with respect to the second targeting probe of the disclosure can mean the amount of active agent alone or in combination with other therapies that provide a therapeutic benefit in the treatment or management of the disease, which can include a decrease in the severity of disease symptoms, an increase in frequency and duration of disease symptom-free periods, or a prevention of impairment or disability due to the disease affliction. The term can encompass an amount that improves overall therapy, reduces or avoids unwanted effects, or enhances the therapeutic efficacy of or synergies with another therapeutic agent.

The term “ratio” or “ratios” as used herein when referring to relative amounts of two or more agents refers to relative amounts of these agents not limited to mole ratios (e.g., mole/mole), weight ratios (w/w, e.g., nanogram/nanogram, μg/μg, ng/μg), volume ratios (v/v, e.g., mL/mL), or weight/volume (w/v, e.g., μg/μL, mg/mL).

As used herein, “ocular disorder,” “ophthalmic disease,” “ophthalmic disorder,” and the like include, but are not limited to, any inflammatory ocular disorders (e.g., dry eye syndrome, glaucoma, cataracts, leucoma, or retinal degeneration) in a subject in need of such treatment comprising administering, to the subject, an effective amount of a compound as set forth above.

As used herein, the terms “prevent,” “preventing,” or “prevention,” “prophylactic treatment,” and the like refer to reducing the probability of developing a disorder or condition in a subject who does not have but is at risk of or susceptible to developing a disorder or condition. The prevention can be complete (i.e., no detectable symptoms) or partial so that fewer symptoms are observed than would likely occur absent treatment. The terms further include a prophylactic benefit. For disease or condition to be prevented, the compositions can be administered to a patient at risk of developing a particular disease or to a patient reporting one or more of the physiological symptoms of a disease, even though a diagnosis of this disease cannot have been made.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either endpoint of the range are specifically contemplated. For example, a nested sub-range of an exemplary range of 1 to 50 can include 1 to 10, 1 to 20, 1 to 30, and 1 to 40 in one direction, or 50 to 40, 50 to 30, 50 to 20, and 50 to 10 in the other direction.

A “subject” may be a human, or a non-human animal not limited to a non-human primate, a dog, a cat, a horse, a rodent, a cow, a goat, a rabbit, and a mouse.

The terms “treat,” “treating,” or “treatment,” and other grammatical equivalents as used herein include alleviating, abating, ameliorating, or preventing a disease, condition or symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit and/or a prophylactic benefit. Therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder.

### 2. Ophthalmic Compositions

The presently disclosed subject matter provides ophthalmic compositions for treating ocular disorders.

2.1. Compositions Comprising IL-4 and/or IL-13.

The present disclosure is directed to a composition comprising an effective amount of interleukin-4 (IL-4). The present disclosure is also directed to a composition comprising an effective amount of interleukin-13 (IL-13). The present disclosure is also directed to a composition comprising effective amounts of a combination of IL-4 and IL-13. In one embodiment, in one aspect, the composition comprises an effective amount of interleukin-4 (IL-4) that can induce cellular activities from various cells and/or tissues to reduce inflammation and/or treat ocular disorders. In another aspect, the composition comprises an effective amount of interleukin-13 (IL-13) that can induce cellular activities from various cells and/or tissues to reduce inflammation and/or treat ocular disorders. In a third aspect, the composition comprises effective amounts of IL-4 and IL-13 that can induce cellular activities from various cells and/or tissues to reduce inflammation and/or treat ocular disorders. For example, in either aspect, the IL-4 and/or IL-13 can reduce neutrophil chemotaxis, and/or decrease the production of reactive oxygen species and/or decrease the production of neutrophil extracellular traps (NET). In macrophages, the IL-4 and/or IL-13 can induce phenotype switching from an M1-pro-inflammatory phenotype to an M2-anti-inflammatory phenotype. In T cells, the IL-4 and/or IL-13 can induce naïve CD4+ cells to differentiate into Th2 cells, which in turn produce additional IL-4 thus helping regulate the inflammatory response and restore homeostasis (20, 33). These cell types have an elevated presence within the ocular surface in DED patients and play a role in perpetuating the pro-inflammatory environment. Thus, restoration of IL-4 and/or IL-13 by employing the disclosed compositions can modulate and shift the Th1/M1-pro-inflammatory environment to a Th2/M2-anti-inflammatory environment, which is characterized by restoring the homeostatic immune environment of the eye.

In other embodiments, the disclosed IL-4 and/or IL-13 can induce interactions between goblet cells and immune cells. Within the ocular epithelium, resident immune cells reside in close proximity to mucin producing goblet cells (22). Goblet-immune cell interaction can be important for ocular surface homeostasis and maintenance of a stable tear film to provide protection against a variety of environmental, microbial, and inflammatory insults. Dysregulation of such ocular immune constituents can affect goblet cell and epithelial cell integrity. For example, inflammatory cytokines produced in DED can induce goblet cell dysfunction and/or death (23). The resulting reduction in mucin production can cause disruption of the tear film, leading to hyperosmolarity and further inflammation. Accordingly, a failure to effectively address both inflammation and goblet cell dysfunction/loss concurrently could be a reason for the limited efficacy of the current therapies.

In yet other embodiments, the disclosed IL-4 and/or IL-13 can induce cellular activities through the IL-4 receptors. For example, the ocular epithelium and goblet cells express the IL-4 receptor. By inducing differentiation of epithelial cells into goblet cells thereby inducing mucin expression, the disclosed IL-4 and/or IL-13 composition can concurrently target both immune cells and ocular cells central to tear film formation to restore homeostasis in ocular disorder patients.

In certain embodiments, the composition can include a clinically relevant concentration of IL-4 and/or IL-13. For example, but not by way of limitation, the concentration of the active agent IL-4 can be from about 0.1 ng to about 10 mg, from about 0.1 ng to about 5 mg, from about 0.1 ng to about 1 mg, from about 0.1 ng to about 1000 μg, from about 0.1 ng to about 500 μg, from about 0.1 ng to about 250 μg, from about 0.1 ng to about 100 μg, from about 0.1 ng to about 50 μg, from about 0.1 ng to about 25 μg, from about 0.1 ng to about 10 μg, or from about 0.1 ng to about 5 μg, from about 0.1 ng to about 1000 ng, from about 0.1 ng to about 500 ng, from about 0.1 ng to about 250 ng, from about 0.1 ng to about 100 ng, from about 0.1 ng to about 50 ng, from about 0.1 ng to about 25 ng, from about 0.1 ng to about 10 ng, from about 0.1 ng to about 5 ng, from about 0.1 ng to about 4 ng, from about 0.1 ng to about 3 ng, from about 0.1 ng to about 2 ng, from about 0.1 ng to about 1 ng, from about 0.5 ng to about 1 ng, or from about 0.01 ng to about 1 ng. Also, for example, but not by way of limitation, the concentration of the active agent IL-13 can be from about 0.1 ng to about 10 mg, from about 0.1 ng to about 5 mg, from about 0.1 ng to about 1 mg, from about 0.1 ng to about 1000 μg, from about 0.1 ng to about 500 μg, from about 0.1 ng to about 250 μg, from about 0.1 ng to about 100 μg, from about 0.1 ng to about 50 μg, from about 0.1 ng to about 25 μg, from about 0.1 ng to about 10 μg, or from about 0.1 ng to about 5 μg, from about 0.1 ng to about 1000 ng, from about 0.1 ng to about 500 ng, from about 0.1 ng to about 250 ng, from about 0.1 ng to about 100 ng, from about 0.1 ng to about 50 ng, from about 0.1 ng to about 25 ng, from about 0.1 ng to about 10 ng, from about 0.1 ng to about 5 ng, from about 0.1 ng to about 4 ng, from about 0.1 ng to about 3 ng, from about 0.1 ng to about 2 ng, from about 0.1 ng to about 1 ng, from about 0.5 ng to about 1 ng, or from about 0.01 ng to about 1 ng.

In certain embodiments, the IL-4 and/or IL-13 can be a lyophilized IL-4 and/or lyophilized IL-13. For example, the disclosed IL-4 can include lyophilized human IL-4, lyophilized rabbit IL-4, lyophilized mouse IL-4, lyophilized monkey IL-4, lyophilized bovine IL-4, or combinations thereof, and the disclosed IL-13 can include lyophilized human IL-13, lyophilized rabbit IL-13, lyophilized mouse IL-13, lyophilized monkey IL-13, lyophilized bovine IL-13, or combinations thereof. In this embodiment, in one aspect, the IL-4 can be an isolated from a natural source. In another aspect, the IL-4 is a recombinant IL-4 produced using prokaryotic or eukaryotic expression host systems including, but not limited to mammalian cells, bacteria, yeast, insect cells, and transgenic plants. Further, the IL-4 can be a full length wild type IL-4 sequence or can be a truncated IL-4 sequence that is capable of performing the functions discussed above. Further in this embodiment, in one aspect, the IL-13 can be an isolated from a natural source. In another aspect, the IL-13 is a recombinant IL-13 produced using prokaryotic or eukaryotic expression host systems including, but not limited to mammalian cells, bacteria, yeast, insect cells, and transgenic plants. Further, the IL-13 can be a full length wild type IL-13 sequence or can be a truncated IL-13 sequence that is capable of performing the functions discussed above.

### 2.1.1 Dermatan Sulfate as a Complexing Agent.

In certain embodiments, the disclosed composition can include dermatan sulfate (DS, also known as chondroitin sulfate B), where the IL-4 and/or IL-13 are each complexed to the dermatan sulfate to form a IL-4/dermatan sulfate complex, a IL-13/dermatan sulfate complex, or a IL-4/IL-13/dermatan sulfate complex. The ability of dermatan sulfate to modulate extracellular matrix (ECM) can enhance bioactivity of IL-4 and/or IL-13. In non-limiting embodiments, the composition can include an effective amount of the dermatan sulfate to enhance the bioactivity of the IL-4 and/or IL-13. For example, but not by way of limitation, the concentration of the dermatan sulfate can be from about 1 ng to about 60 mg, from about 1 ng to about 50 mg, from about 1 ng to about 40 mg, from about 1 ng to about 30 mg, from about 1 ng to about 20 mg, from about 1 ng to about 10 mg, from about 1 ng to about 6000 μg, from about 1 ng to about 5000 μg, from about 1 ng to about 1000 μg, from about 1 ng to about 500 μg, from about 1 ng to about 250 μg, from about 1 ng to about 120 μg, from about 1 ng to about 100 μg, from about 1 ng to about 50 μg, from about 100 ng to about 60 mg, from about 100 ng to about 50 mg, from about 100 ng to about 40 mg, from about 100 ng to about 30 mg, from about 100 ng to about 20 mg, from about 100 ng to about 10 mg, from about 100 ng to about 6000 μg, from about 100 ng to about 5000 μg, from about 100 ng to about 1000 μg, from about 100 ng to about 500 μg, from about 100 ng to about 250 μg, from about 100 ng to about 120 μg, from about 100 ng to about 100 μg, or from about 100 ng to about 50 μg.

In certain embodiments, the IL-4 and/or IL-13 can be complexed with the disclosed dermatan sulfate at a pre-determined ratio. IL-4 and/or IL-13 can be complexed with the dermatan sulfate, because they have a relatively high isoelectric point and are therefore positively charged at physiologic pH, rendering its binding to negatively charged groups in dermatan sulfate. By complexing with the IL-4 and IL-13 in an effective ratio, the dermatan sulfate can facilitate, enhance, and support certain cellular signaling functions of IL-4 and IL-13. In non-limiting embodiments, the ratio of the IL-4 to the dermatan sulfate can be from about 1:1000 to about 1:120000, from about 1:1000 to about 1:100000, from about 1:1000 to about 1:60000, from about 1:1000 to about 1:50000, from about 1:1000 to about 1:40000, from about 1:1000 to about 1:40000, from about 1:1000 to about 1:30000, from about 1:1000 to about 1:20000, or from about 1:1000 to about 1:10000 (all ratios are w/w). In non-limiting embodiments, the ratio of the IL-13 to the dermatan sulfate can be from about 1:1000 to about 1:120000, from about 1:1000 to about 1:100000, from about 1:1000 to about 1:60000, from about 1:1000 to about 1:50000, from about 1:1000 to about 1:40000, from about 1:1000 to about 1:40000, from about 1:1000 to about 1:30000, from about 1:1000 to about 1:20000, or from about 1:1000 to about 1:10000 (all ratios are w/w).

In certain embodiments, the disclosed composition can include a solvent. The solvent can be either aqueous or non-aqueous. For example, but not by way of limitation, the solvent can include sterile water, polyvinyl alcohol (PVA), hyaluronic acid, glycerin, dermatan sulfate, methycellulose, gellan gum, xanthan gum, trehalose, or a combination of these. In certain embodiments, the composition can also include a buffer including, but not limited to an acetate buffer, a phosphate buffer, a citrate buffer, and a glutamate buffer. For example, the composition can include phosphate buffered saline (PBS) with or without trehalose.

In certain embodiments, the composition can include a pharmaceutically acceptable carrier. The pharmaceutically acceptable carrier can include a physiologically compatible vehicle. In non-limiting embodiments, the pharmaceutically acceptable carrier can include a carrier protein including, but not limited to an albumin or a gelatin. For example, the disclosed composition can include bovine serum albumin (BSA) as a carrier protein. In non-limiting embodiments, the carrier is present in the composition in a concentration from about 0.1 mg/mL to about 1 mg/mL, from about 0.5 mg/mL to about 1 mg/mL, from about 1 mg/mL to about 1.5 mg/mL, from about 1.0 mg/mL to about 2 mg/mL, from about 2 mg/mL to about 3 mg/mL, from about 3 mg/mL to about 4 mg/mL, from about 4 mg/mL to about 5 mg/mL, from about 5 mg/mL to about 6 mg/mL, from about 6 mg/mL to about 7 mg/mL, from about 7 mg/mL to about 8 mg/mL, from about 8 mg/mL to about 9 mg/mL, from about 9 mg/mL to about 10 mg/mL (i.e., 1% w/v), from about 0.5 mg/mL to about 10 mg/mL, from about 10 mg/mL to about 15 mg/mL, and from about 15 mg/mL to about 20 mg/mL.

In certain embodiments, the composition can be formulated as a solution, gel, foam, ointment, microemulsion, in situ gel, contact lens coating, ocular insert, minidisc, soluble ophthalmic drug insert, minitablet, microparticle, nanoparticle, liposome, or spray. For example, the formulated solution can be administered to the eye of the subject for contacting the composition with the eye.

In certain embodiments, the composition can be formulated as an eye drop solution. In some embodiments, the eye drop solution can include an effective amount of the IL-4 ranging from about 1 ng of the IL-4 per 5 μL of the solution to about 1 ng of the IL-4 per 500 μL of the solution. In non-limiting embodiments, the eye drop solution can include a concentration of 1 ng IL-4 per 30 μL of the solution. In non-limiting embodiments, the eye drop solution can include the effective amount of the dermatan sulfate ranging from about 1 mg/ml to about 10 mg/ml. In another embodiment, the eye drop solution can include an effective amount of the IL-13 ranging from about 1 ng of the IL-13 per 5 μL of the solution to about 1 ng of the IL-13 per 500 μL of the solution. In non-limiting embodiments, the eye drop solution can include a concentration of 1 ng IL-13 per 30 μL of the solution. In non-limiting embodiments, the eye drop solution can include the effective amount of the dermatan sulfate ranging from about 1 mg/ml to about 10 mg/ml. In yet another embodiment, the eye drop solution can include an effective amount of the IL-4 and IL-13 ranging from about 1 ng each of the IL-4 and IL-13 per 5 μL of the solution to about 1 ng of each of the IL-4 and IL-13 per 500 L of the solution. In non-limiting embodiments, the eye drop solution can include a concentration of 1 ng each of IL-4 and IL-13 per 30 μL of the solution. In non-limiting embodiments, the eye drop solution can include the effective amount of the dermatan sulfate ranging from about 1 mg/ml to about 10 mg/ml.

In certain embodiments, the composition can be formulated into a unit dosage form to provide a total daily dosage and can be suitably filled in a container, which can enable the quantitative administration of the composition. The total daily dosage can be various based on the target tissue (e.g., size of damaged tissue) or patient. For this purpose, the composition can be formulated to be used once or several times. For example, several divided doses can be administered daily. In non-limiting embodiments, two drops of the disclosed eye drop solution can be administered into an eye of a subject daily or twice daily to deliver a dose of 2 ng of the IL-4 or the IL-13. Alternatively, two drops of the disclosed eye drop solution can be administered into an eye of a subject daily or twice daily to deliver a dose of 2 ng of each of the IL-4 and the IL-13.

In certain embodiments, in some aspects, the composition for treating an ocular disorder can include an effective amount of IL-4 and dermatan sulfate. In other aspects, the composition for treating an ocular disorder can include an effective amount of IL-13 and dermatan sulfate. In yet another aspect, the composition for treating an ocular disorder can include an effective amount of IL-4 and dermatan sulfate, and IL-13 and dermatan sulfate. The IL-4 and/or IL-13 can be complexed with the dermatan sulfate in a ratio from about 1:1000 to about 1:120000. In non-limiting embodiments, the ratio can be about 1:60000. The concentration of the IL-4 can range from about 0.1 ng to about 50 ng, and the concentration of the dermatan sulfate can range from about 100 ng to about 6000 μg. In non-limiting embodiments, the disclosed ophthalmic composition can be formulated in the form of an eye drop solution. The eye drop solution can further include a buffer (e.g., PBS) with a carrier protein (e.g., bovine serum albumin). In non-limiting embodiments, the eye drop solution can include a concentration of about 1 ng of the IL-4 and/or 1 ng of the IL-13 per 5 μL of the solution to about 1 ng of the IL-4 and/or 1 ng of the IL-13 per 500 μL of the solution. In non-limiting embodiments, the eye drop solution can include a concentration of about 2 mg/mL of dermatan sulfate.

### 2.2. Compositions Comprising Interleukin-4 Receptor Signaling Activators.

The present disclosure is also directed to a composition comprising effective amounts of at least one activator of interleukin-4 receptor (IL-4R) signaling (“activator”). In this embodiment, the activator of IL-4R signaling is any agent or compound that activates IL-4R dependent signaling pathways. In this embodiment, the IL-4R receptor comprises one or more IL-4R subtypes. For example and not by any way of limitation, the disclosed composition comprises activators that activates IL-4R Type I and/or IL-4R Type II and/or IL-4R Type III.

In this embodiment, in one aspect, the at least one activator of IL-4R signaling can comprise interleukin-4 (IL-4) that can induce cellular activities from various cells and/or tissues to reduce inflammation and/or treat ocular disorders. In another aspect, the activator can comprise interleukin-13 (IL-13) that can induce cellular activities from various cells and/or tissues to reduce inflammation and/or treat ocular disorders. In a third aspect, the activator can comprise a combination of IL-4 and IL-13 that can induce cellular activities from various cells and/or tissues to reduce inflammation and/or treat ocular disorders. In certain embodiments, the disclosed composition can include dermatan sulfate (DS, also known as chondroitin sulfate B), where the IL-4 and/or IL-13 are each complexed to the dermatan sulfate to form an IL-4/dermatan sulfate complex, or an IL-13/dermatan sulfate complex, or a combination thereof, or an IL-4/IL-13/dermatan sulfate complex.

Details relating to compositions comprising uncomplexed IL-4, IL-13 or, a combination of IL-4 and IL-13 and, compositions comprising dermatan sulfate complexed IL-4, IL-13 or, a combination thereof, including a disclosure of solvents and carriers are presented in detail in the description of embodiments in the previous sections.

In non-limiting examples, the at least one activator of IL-4R signaling can be IL-4, IL-13, or a combination thereof, which can reduce neutrophil chemotaxis, and/or decrease the production of reactive oxygen species and/or decrease the production of neutrophil extracellular traps (NET). In macrophages, the at least one activator of IL-4R signaling can induce phenotype switching from an M1-pro-inflammatory phenotype to an M2-anti-inflammatory phenotype. In T cells, the at least one activator of IL-4R signaling can induce naïve CD4+ cells to differentiate into Th2 cells, which in turn produce additional IL-4 thus helping regulate the inflammatory response and restore homeostasis (20, 33). These cell types have an elevated presence within the ocular surface in DED patients and play a role in perpetuating the pro-inflammatory environment. Thus, restoration of IL-4 and/or IL-13 by employing the disclosed compositions can modulate and shift the Th1/M1-pro-inflammatory environment to a Th2/M2-anti-inflammatory environment, which is characterized by restoring the homeostatic immune environment of the eye.

In other embodiments, the at least one activator of IL-4R signaling can induce interactions between goblet cells and immune cells. Within the ocular epithelium, resident immune cells reside in close proximity to mucin producing goblet cells (22). Goblet-immune cell interaction can be important for ocular surface homeostasis and maintenance of a stable tear film to provide protection against a variety of environmental, microbial, and inflammatory insults. Dysregulation of such ocular immune constituents can affect goblet cell and epithelial cell integrity. For example, inflammatory cytokines produced in DED can induce goblet cell dysfunction and/or death (23). The resulting reduction in mucin production can cause disruption of the tear film, leading to hyperosmolarity and further inflammation. Accordingly, a failure to effectively address both inflammation and goblet cell dysfunction/loss concurrently could be a reason for the limited efficacy of the current therapies.

In yet other embodiments, the at least one activator of IL-4R signaling can induce cellular activities through the IL-4 receptors. For example, the ocular epithelium and goblet cells express the IL-4 receptor. By inducing differentiation of epithelial cells into goblet cells thereby inducing mucin expression, the disclosed composition can concurrently target both immune cells and ocular cells central to tear film formation to restore homeostasis in ocular disorder patients.

In certain embodiments, the composition can include a clinically relevant concentration of the activator of IL-4R signaling. For example, but not by way of limitation, the concentration of the activator can be from about 0.1 ng to about 10 mg, from about 0.1 ng to about 5 mg, from about 0.1 ng to about 1 mg, from about 0.1 ng to about 1000 μg, from about 0.1 ng to about 500 μg, from about 0.1 ng to about 250 μg, from about 0.1 ng to about 100 μg, from about 0.1 ng to about 50 μg, from about 0.1 ng to about 25 μg, from about 0.1 ng to about 10 μg, or from about 0.1 ng to about 5 μg, from about 0.1 ng to about 1000 ng, from about 0.1 ng to about 500 ng, from about 0.1 ng to about 250 ng, from about 0.1 ng to about 100 ng, from about 0.1 ng to about 50 ng, from about 0.1 ng to about 25 ng, from about 0.1 ng to about 10 ng, from about 0.1 ng to about 5 ng, from about 0.1 ng to about 4 ng, from about 0.1 ng to about 3 ng, from about 0.1 ng to about 2 ng, from about 0.1 ng to about 1 ng, from about 0.5 ng to about 1 ng, or from about 0.01 ng to about 1 ng.

In certain embodiments, the activator can be in a lyophilized form. In certain embodiments, the activator can be isolated from natural sources not limited to human, mouse, rabbit, monkey, bovine, or combinations thereof. In other embodiments the activator can be obtained using synthetic methods. In certain embodiments the activator is obtained by recombinant technology using prokaryotic or eukaryotic expression host systems including, but not limited to mammalian cells, bacteria, yeast, insect cells, and transgenic plants. Further, where the activator is a protein, it can have a full length, wild type sequence or can have a truncated sequence that is capable of performing the functions discussed above.

### 2.2.1 Dermatan Sulfate as a Complexing Agent.

In certain embodiments, the disclosed composition can include dermatan sulfate (DS, also known as chondroitin sulfate B), where the activator is complexed with the dermatan sulfate. The ability of dermatan sulfate to modulate extracellular matrix (ECM) can enhance bioactivity of the activator. In non-limiting embodiments, the composition can include an effective amount of the dermatan sulfate to enhance the bioactivity of the activator. For example, but not by way of limitation, the concentration of the dermatan sulfate can be from about 1 ng to about 60 mg, from about 1 ng to about 50 mg, from about 1 ng to about 40 mg, from about 1 ng to about 30 mg, from about 1 ng to about 20 mg, from about 1 ng to about 10 mg, from about 1 ng to about 6000 μg, from about 1 ng to about 5000 μg, from about 1 ng to about 1000 μg, from about 1 ng to about 500 μg, from about 1 ng to about 250 μg, from about 1 ng to about 120 μg, from about 1 ng to about 100 μg, from about 1 ng to about 50 μg, from about 100 ng to about 60 mg, from about 100 ng to about 50 mg, from about 100 ng to about 40 mg, from about 100 ng to about 30 mg, from about 100 ng to about 20 mg, from about 100 ng to about 10 mg, from about 100 ng to about 6000 μg, from about 100 ng to about 5000 μg, from about 100 ng to about 1000 μg, from about 100 ng to about 500 μg, from about 100 ng to about 250 μg, from about 100 ng to about 120 μg, from about 100 ng to about 100 μg, or from about 100 ng to about 50 μg.

In certain embodiments, the activator can be complexed with the disclosed dermatan sulfate at a pre-determined ratio. Complexation with the dermatan sulfate is enabled when the activator has a relatively high isoelectric point and thereby positively charged at physiologic pH, rendering its binding to negatively charged groups in dermatan sulfate. By complexing with the activator in an effective ratio, the dermatan sulfate can facilitate, enhance, and support certain cellular signaling functions of attributable to the activator. In non-limiting embodiments, the ratio of the activator to the dermatan sulfate can be from about 1:1000 to about 1:120000, from about 1:1000 to about 1:100000, from about 1:1000 to about 1:60000, from about 1:1000 to about 1:50000, from about 1:1000 to about 1:40000, from about 1:1000 to about 1:40000, from about 1:1000 to about 1:30000, from about 1:1000 to about 1:20000, or from about 1:1000 to about 1:10000 (all ratios are w/w).

In certain embodiments, the disclosed composition can include a solvent. The solvent can be either aqueous or non-aqueous. For example, but not by way of limitation, the solvent can include sterile water, polyvinyl alcohol (PVA), hyaluronic acid, glycerin, dermatan sulfate, methycellulose, gellan gum, xanthan gum, trehalose, or a combination of these. In certain embodiments, the composition can also include a buffer including, but not limited to an acetate buffer, a phosphate buffer, a citrate buffer, and a glutamate buffer. For example, the composition can include phosphate buffered saline (PBS) with or without trehalose.

In certain embodiments, the composition can include a pharmaceutically acceptable carrier. The pharmaceutically acceptable carrier can include a physiologically compatible vehicle. In non-limiting embodiments, the pharmaceutically acceptable carrier can include a carrier protein including, but not limited to an albumin or a gelatin. For example, the disclosed composition can include bovine serum albumin (BSA) as a carrier protein. In non-limiting embodiments, the carrier is present in the composition in a concentration from about 0.1 mg/mL to about 1 mg/mL, from about 0.5 mg/mL to about 1 mg/mL, from about 1 mg/mL to about 1.5 mg/mL, from about 1.0 mg/mL to about 2 mg/mL, from about 2 mg/mL to about 3 mg/mL, from about 3 mg/mL to about 4 mg/mL, from about 4 mg/mL to about 5 mg/mL, from about 5 mg/mL to about 6 mg/mL, from about 6 mg/mL to about 7 mg/mL, from about 7 mg/mL to about 8 mg/mL, from about 8 mg/mL to about 9 mg/mL, from about 9 mg/mL to about 10 mg/mL (i.e., 1% w/v), from about 0.5 mg/mL to about 10 mg/mL, from about 10 mg/mL to about 15 mg/mL, and from about 15 mg/mL to about 20 mg/mL.

In certain embodiments, the composition can be formulated as a solution, gel, foam, ointment, microemulsion, in situ gel, contact lens coating, ocular insert, minidisc, soluble ophthalmic drug insert, minitablet, microparticle, nanoparticle, liposome, or spray. For example, the formulated solution can be administered to the eye of the subject for contacting the composition with the eye.

In certain embodiments, the composition can be formulated as an eye drop solution. In some embodiments, the eye drop solution can include an effective amount of the activator ranging from about 1 ng of the activator per 5 μL of the solution to about 1 ng of the activator per 500 μL of the solution. In non-limiting embodiments, the eye drop solution can include a concentration of 1 ng activator per 30 μL of the solution. In non-limiting embodiments, the eye drop solution can include the effective amount of the dermatan sulfate ranging from about 1 mg/ml to about 10 mg/ml.

In certain embodiments, the composition can be formulated into a unit dosage form to provide a total daily dosage and can be suitably filled in a container, which can enable the quantitative administration of the composition. The total daily dosage can be various based on the target tissue (e.g., size of damaged tissue) or patient. For this purpose, the composition can be formulated to be used once or several times. For example, several divided doses can be administered daily. In non-limiting embodiments, two drops of the disclosed eye drop solution can be administered into an eye of a subject daily or twice daily to deliver a dose of 2 ng of the activator.

In certain embodiments, the composition for treating an ocular disorder can include an effective amount of the activator and dermatan sulfate. The activator can be complexed with the dermatan sulfate in a ratio from about 1:1000 to about 1:120000. In non-limiting embodiments, the ratio can be about 1:60000. The concentration of the IL-4 can range from about 0.1 ng to about 50 ng, and the concentration of the dermatan sulfate can range from about 100 ng to about 6000 μg. In non-limiting embodiments, the disclosed ophthalmic composition can be formulated in the form of an eye drop solution. The eye drop solution can further include a buffer (e.g., PBS) with a carrier protein (e.g., bovine serum albumin). In non-limiting embodiments, the eye drop solution can include a concentration of about 1 ng of the activator per 5 μL of the solution to about 1 ng of the activator per 500 μL of the solution. In non-limiting embodiments, the eye drop solution can include a concentration of about 2 mg/mL of dermatan sulfate.

### 3. Methods of Manufacturing the Ophthalmic Composition

The presently disclosed subject matter also relates to methods for manufacturing an ophthalmic composition for treating ocular disorders.

3.1. Compositions Comprising IL-4 and/or IL-13.

In a non-limiting embodiment, the ophthalmic composition can be manufactured as an eye drop solution, a suspension, an ointment, or spray. In certain embodiments, the ophthalmic composition is an eye drop solution.

In this embodiment, in one aspect, the ophthalmic composition can be an eye drop solution including an effective amount of IL-4. In another aspect, the ophthalmic composition can be an eye drop solution including an effective amount of IL-13. In a third aspect, the ophthalmic composition can be an eye drop solution including effective amounts of a combination of IL-4 and IL-13. For example, in either aspect, to make the IL-4 and/or IL-13 eye drops for treating ocular disorders, lyophilized IL-4 (e.g., human IL-4) and/or lyophilized IL-13 (e.g., human IL-13) can be reconstituted in sterile solvent (e.g., PBS) with a carrier protein (e.g., bovine serum albumin). In non-limiting embodiments, the final concentration of the eye drop solution can be about 1 ng IL-4 per 30 μL and/or about 1 ng IL-13 per 30 μL so that when two drops (˜60 μL) are given, a dose of 2 ng can be delivered to the ocular surface.

In certain embodiments, the ophthalmic composition can be an eye drop solution including an effective amount of IL-4 and dermatan sulfate and/or an effective amount of IL-13 and dermatan sulfate. For example, to make the IL-4 and/or IL-13 eye drops for treating ocular disorders, lyophilized IL-4 (e.g., human IL-4) and/or lyophilized IL-13 (e.g., human IL-13) can be reconstituted in sterile solvent (e.g., PBS) with a carrier protein (e.g., bovine serum albumin). In non-limiting embodiments, the final concentration of the eye drop solution can be about 1 ng IL-4 and/or about 1 ng IL-13 per 30 μL so that when two drops (˜60 μL) are given, a dose of 2 ng can be delivered to the ocular surface. In non-limiting embodiments, the carrier is present in the composition in a concentration from about 0.1 mg/mL to about 1 mg/mL, from about 0.5 mg/mL to about 1 mg/mL, from about 1 mg/mL to about 1.5 mg/mL, from about 1.0 mg/mL to about 2 mg/mL, from about 2 mg/mL to about 3 mg/mL, from about 3 mg/mL to about 4 mg/mL, from about 4 mg/mL to about 5 mg/mL, from about 5 mg/mL to about 6 mg/mL, from about 6 mg/mL to about 7 mg/mL, from about 7 mg/mL to about 8 mg/mL, from about 8 mg/mL to about 9 mg/mL, from about 9 mg/mL to about 10 mg/mL (i.e., 1% w/v), from about 0.5 mg/mL to about 10 mg/mL, from about 10 mg/mL to about 15 mg/mL, and from about 15 mg/mL to about 20 mg/mL.

In certain embodiments, the IL-4 and/or the IL-13 can be complexed with dermatan sulfate in a pre-determined ratio that can enhance the bioactivity of the IL-4 and/or the IL-13. For example, the reconstituted IL-4 and/or IL-13 can be added to a 2 mg/mL solution of dermatan sulfate in a solvent (e.g., PBS) with a carrier protein (e.g., bovine serum albumin), or in distilled water to give a final concentration of 1 ng IL-4 and/or about 1 ng IL-13 per 30 L of solution. The mixture can be incubated (e.g., overnight at 4° C.) to allow dermatan sulfate to form complexes with the IL-4 and/or the IL-13. In non-limiting embodiments, the eye drop solution can be formulated in a way that, when two drops are given, about 2 ng of IL-4 and/or 2 ng of IL-13 complexed within 120 μg of dermatan sulfate can be delivered.

### 3.2. Compositions Comprising Interleukin-4 Receptor Signaling Activators.

In a non-limiting embodiment, the ophthalmic composition can be manufactured as an eye drop solution, a suspension, an ointment, or spray. In certain embodiments, the ophthalmic composition is an eye drop solution. In this embodiment, in one aspect, the ophthalmic composition can be an eye drop solution comprising effective amounts of at least one activator of interleukin-4 receptor (IL-4R) signaling. In this embodiment, the activator of IL-4R signaling is any agent or compound that activates IL-4R dependent signaling pathways. In this embodiment, the IL-4R receptor comprises one or more IL-4R subtypes. For example and not by any way of limitation, the disclosed composition comprises activators that activates IL-4R Type I and/or IL-4R Type II and/or IL-4R Type III.

In this embodiment, in one aspect, the activator can comprise interleukin-4 (IL-4) that can induce cellular activities from various cells and/or tissues to reduce inflammation and/or treat ocular disorders. In another aspect, the activator can comprise interleukin-13 (IL-13) that can induce cellular activities from various cells and/or tissues to reduce inflammation and/or treat ocular disorders. In a third aspect, the activator can comprise a combination of IL-4 and IL-13 that can induce cellular activities from various cells and/or tissues to reduce inflammation and/or treat ocular disorders. In certain embodiments, the disclosed composition can include dermatan sulfate (DS, also known as chondroitin sulfate B), where the IL-4 and/or IL-13 are each complexed to the dermatan sulfate to form a IL-4/dermatan sulfate complex, a IL-13/dermatan sulfate complex, or a IL-4/IL-13/dermatan sulfate complex. Details relating to manufacturing of the ophthalmic compositions comprising uncomplexed IL-4, IL-13 or, a combination of IL-4 and IL-13 and, compositions comprising dermatan sulfate complexed IL-4, IL-13 or, a combination thereof, including disclosure of solvents and carriers are presented in detail in the description of embodiments in the previous sections.

In certain embodiments, to make the activator composition eye drops for treating ocular disorders, lyophilized activator can be reconstituted in sterile solvent (e.g., PBS) with a carrier protein (e.g., bovine serum albumin). In non-limiting embodiments, the final concentration of the eye drop solution can be about 1 ng activator per 30 μL so that when two drops (˜60 μL) are given, a dose of 2 ng can be delivered to the ocular surface.

In certain embodiments, the activator can be complexed with dermatan sulfate in a pre-determined ratio that can enhance the bioactivity of the activator. For example, the reconstituted activator can be added to a 2 mg/mL solution of dermatan sulfate in a solvent (e.g., PBS) with a carrier protein (e.g., bovine serum albumin, 1% w/v), or in distilled water to give a final concentration of 1 ng activator per 30 μL of solution. The mixture can be incubated (e.g., overnight at 4° C.) to allow dermatan sulfate to form complex with the activator. In non-limiting embodiments, the eye drop solution can be formulated in a way that, when two drops are given, about 2 ng of activator complexed within 120 μg of dermatan sulfate can be delivered.

### 4. Methods of Treating Ocular Disorders

The presently disclosed subject matter provides a method for treating ocular disorders. In certain non-limiting embodiments, the method comprises administering the disclosed ophthalmic composition to an eye of a subject.

In certain embodiments, the administration of the ophthalmic composition can concurrently improve tear film stability and reduce inflammation. For example, the administration of the disclosed ophthalmic composition can concurrently improve tear film stability and mitigation of symptoms and causes of inflammatory eye diseases by restoring goblet cell number and function. In a non-limiting example, the symptoms of ocular disorders like inflammatory eye diseases include redness, itching, burning, foreign body sensation, watery eyes, dry eyes, swelling, pain, clouding of vision, secretion of pus, sticking eyelids, and/or altered sensitivity to light. In non-limiting embodiments, the effective concentration of IL-4 and/or IL-13, or the effective concentrations of IL-4/dermatan sulfate complex and/or effective concentrations of IL-13/dermatan sulfate complex can be a concentration that alleviates at least one of the symptoms of ocular disorder. In non-limiting embodiments, the effective concentration of the activator of IL-4R signaling or, the activator of IL-4R signaling in a complex with dermatan sulfate can be a concentration that alleviates at least one of the symptoms of ocular disorder. In non-limiting embodiments, the disclosed composition can be an immune cell modulator and a goblet cell modulator.

In non-limiting embodiments, the ocular eye disease is inflammatory eye disease which includes, but is not limited to dry eye syndrome, uveitis (scleritis, myositis, chorioretinitis), infection, allergies, trauma, keratoconjunctivitis sicca, and post-surgical recovery.

In certain embodiments, the disclosed composition can be administered topically to the eye of the subject. In a non-limiting embodiment, the topical administration is of an eye drop solution, a suspension, an ointment, or spray. An example of topical administration can include direct application of the composition in the form of, for example, an eye drop solution to a subject in order to contact same with an eye.

In certain embodiments, the disclosed composition can be formulated in the form of an eye drop solution and administered topically to the eye of the subject. In non-limiting embodiments, the eye drop solution can be administered daily for about one day, about two days, about three days, about four days, about five days, about six days, about seven days, about eight days, about nine days, about ten days, about two weeks, about 3 weeks, or about 4 weeks.

In non-limiting embodiments, the dosage of the eye drop solution can be about 2 drops of solution. Each drop of the composition in a solution can correspond to a volume for example, but not by way of limitation, from about 20 μL to about 30 μL, from about 30 μL to about 40 μL, from about 40 μL to about 50 μL, from about 50 μL to about 60 μL, from about 60 μL to about 70 μL from about 70 μL to about 80 μL, from about 80 μL to about 90 μL, or from about 90 μL to about 100 μL of the eye drop solution. In non-limiting embodiments, each drop of the eye drop solution can correspond to about 0.1 ng to about 50 ng of IL-4 and/or 0.1 ng to about 50 ng of IL-13. For example, when two drops (˜60 μL) are given, a dose of 2 ng of IL-4 and/or 2 ng of IL-13 can be delivered to the ocular surface.

In certain embodiments, the composition can be administered to an eye of a subject by administering the disclosed dosage of the composition to the eye at least one time daily, two times daily, or up to three times daily. In non-limiting embodiments, the composition can be periodically administered (e.g., about once a week or once every two days).

### EXAMPLES

The following examples are provided to further illustrate some embodiments of the present invention but are not intended to limit the scope of the invention; it will be understood by their exemplary nature that other procedures, methodologies, or methods known to those skilled in the art can alternatively be used.

The disclosed subject matter provides a single-cytokine drug delivery method that can simultaneously address both the tear film instability and inflammation that are characteristic of DED, resulting in improved long-term relief of DED compared to currently available treatment options. DED is characterized by a chronic pro-inflammatory environment that leads to tissue damage (7). This pro-inflammatory environment includes elevated levels of pro-inflammatory cytokines and matrix metalloproteases (MMP) within the tear film in human clinical subjects and animal models (15, 16). These increases in pro-inflammatory cytokine levels are accompanied by decreases or loss of multiple anti-inflammatory and regulatory cytokines, including IL-4, within the tear film of DED patients (17). IL-4 is a potent immunomodulatory and regulatory cytokine that reduces neutrophil chemotaxis and production of radical oxygen and neutrophil extracellular traps (18). This role can be particularly beneficial in DED since neutrophil driven chronic inflammation affecting lacrimal glands and ocular surfaces as well as neutrophil extracellular traps are important pathogenic mechanisms of dry eyes, especially in the elderly (31). In macrophages, IL-4 induces transition from the M1-pro-inflammatory phenotype (19, 32). In T-cells, IL-4 induces naïve CD4+ cells to differentiate into Th2 cells, which further regulate the inflammatory response and restore homeostasis (20, 33). Thus, restoration of IL-4 at the ocular surface using low dose, localized therapy in patients with inflammatory dry eye can modulate the inflammatory environment, regulate inflammation, and restore homeostasis without requiring systemic, non-specific, or chronic immune modulation.

Within the ocular epithelium, resident immune cells reside in close proximity to mucin producing goblet cells (22). Goblet-immune cell interaction is critical for ocular surface homeostasis and maintenance of a stable tear film to provide protection against a variety of environmental, microbial, and inflammatory insults (22). Thus, dysregulation of ocular immune constituents has the potential to significantly affect goblet cells and epithelial integrity. The inflammatory cytokines produced in the dry eye have been shown to induce goblet dysfunction and death (23). The resulting reduction in mucin production results in disruption of the tear film, leading to hyperosmolarity and further inflammation. Thus, an understanding and consideration of goblet cell biology and its relationship to immune cell function and epithelial integrity are crucial, and failure to effectively address both inflammation and goblet cell dysfunction/loss concurrently can explain the limited efficacy of current therapeutic options. Interestingly, the ocular epithelium and goblet cells express the IL-4 receptor, and IL-4 has been shown to cause differentiation of epithelial cells into goblet cells and to induce mucin expression (22, 24). Thus, IL-4 can represent a single cytokine capable of concurrently targeting a spectrum of inflammatory, epithelial, and goblet cell constituents of the ocular surface to restore homeostasis in the setting of DED. Indeed, the disclosed showed that it does play a homeostatic role in the ocular surface. IL-4 has been detected in tear film of healthy human subjects and is decreased in DED patients (17), suggesting a homeostatic role. Providing further evidence for IL-4 as a homeostatic molecule within the ocular surface, patients receiving the IL-4R blocker dupilumab to prevent allergic disease frequently experience new or aggravated dry eye as a side effect—a finding that is associated with ocular goblet cell dysfunction, mucin deficiency, and inflammation (34, 35).

### Example 1

### IL-4 Eye Drop Formulation in Treatment of Dry Eye Disease.

This example describes utility of an eye drop composition comprising IL-4 for the treatment of dry eyes.

Formulation 1—A reconstituted solution of IL-4 was prepared by dissolving lyophilized rabbit IL-4 in a sterile solution of bovine serum albumin (carrier protein, 1% w/v) in PBS to obtain a working IL-4 concentration of 1 ng/30 μL (1 drop).

Formulation 2-A second formulation of dermatan sulfate (DS) complexed IL-4 was prepared by reconstituting lyophilized rabbit IL-4 in a sterile solution of dermatan sulfate and bovine serum albumin in PBS (2 mg/ml DS, 1% w/v BSA) to obtain a working IL-4 concentration of 1 ng/30 μL (1 drop).

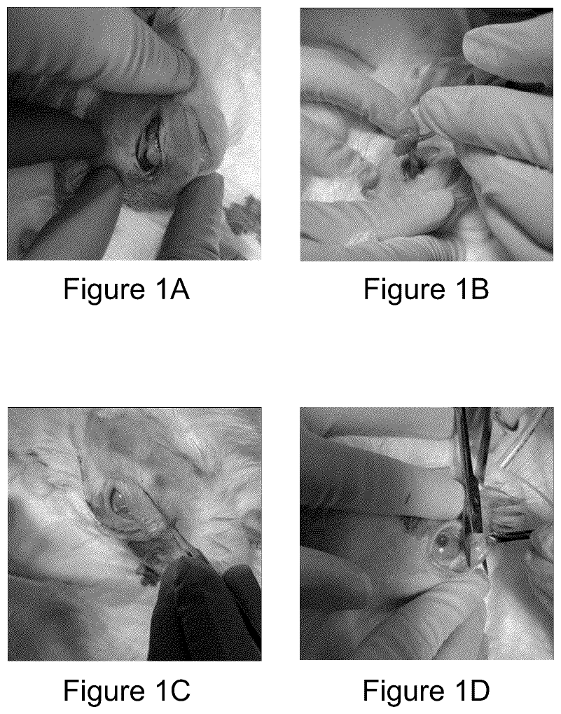

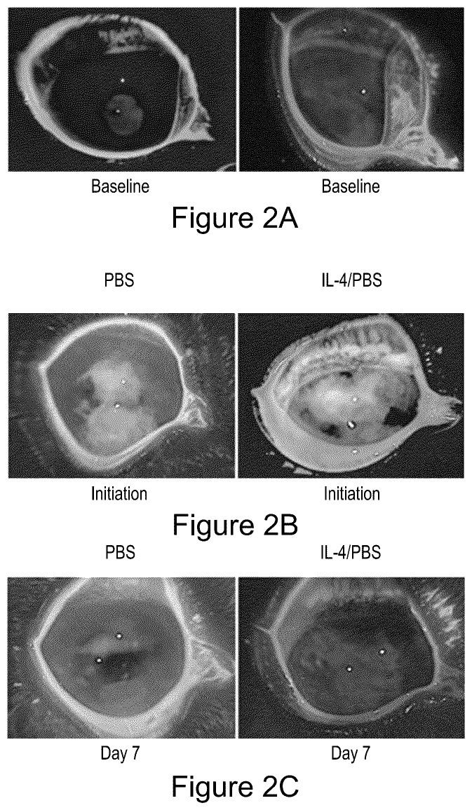

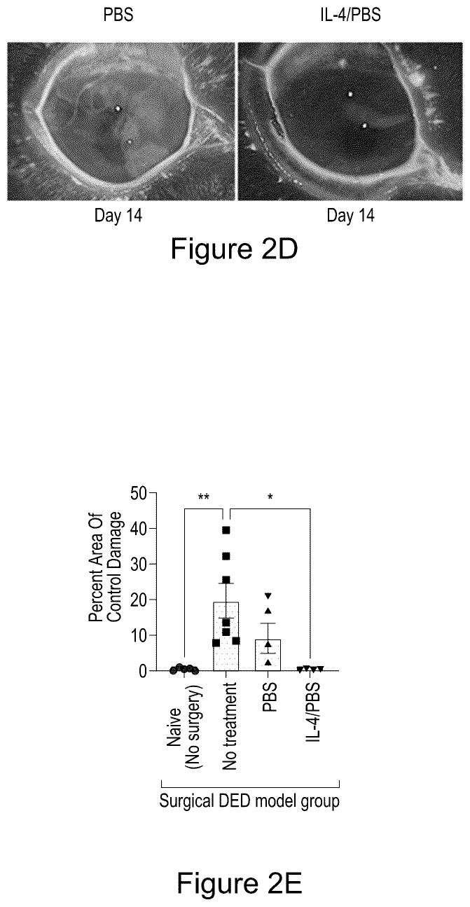

A DED rabbit model was used to test the efficacy of the eye drop formulations. The inferior lacrimal gland and the nictitating membrane were removed from rabbits (FIGS. 1A-1D) and DED allowed to develop for 4 weeks. Development of DED was confirmed by fluorescein staining (FIG. 2B). The animals were then divided into two groups, with the control group getting two drops of PBS and the test group getting two drops of the IL-4 formulation, once daily for 14 days.

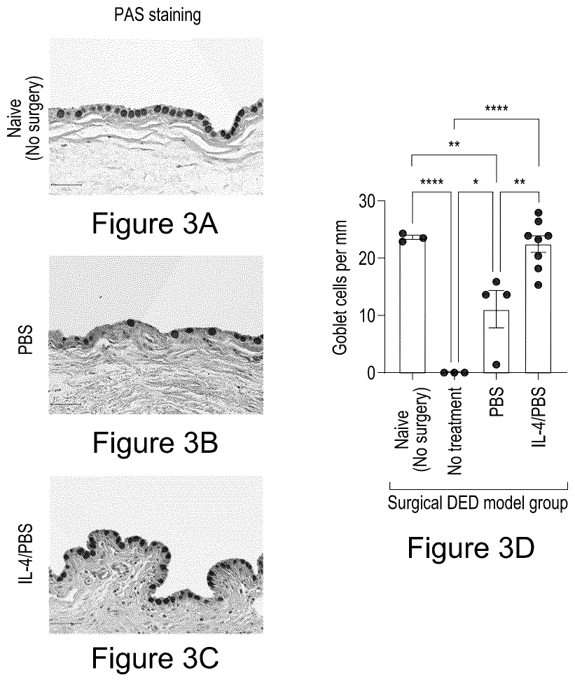

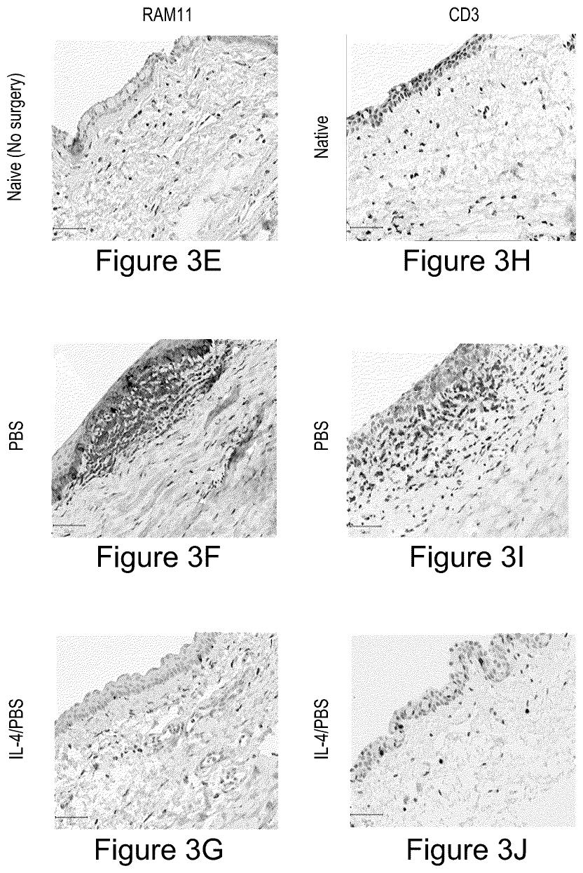

Ocular assessment was performed by fluorescein staining at day 7 and day 14 after initiation of treatment. As evident from FIGS. 2A-2E, significant reductions in fluorescein staining were observed in the IL-4 treated group as compared to the control group. Histological analysis (FIGS. 3A-3D) revealed that administration of the IL-4 formulation was associated with a return in the number of goblet cells to levels observed in control animals (lacrimal gland and the nictitating membrane not removed). Immunostaining for rabbit macrophages using anti-RAM11 antibodies demonstrated significant differences between the PBS and IL4-treated animals (FIGS. 3E-3G). PBS treated animals showed areas of dense RAM11⁺ cell infiltrates in the epithelial thinning indicative of ulceration. Interestingly, elevated RAM11 expression is associated with epithelial wounding, suggesting increased damage to the epithelium in PBS treated animals but not in the IL-4 treated animals. CD3 immunostaining demonstrated presence of T-cells within the same areas as the RAM11 staining, in PBS treated animals (FIG. 3I). However, significantly lower T-cells were observed in the IL-4 treated group (FIG. 3J), suggesting that IL-4 treatment regulates the T-cell response. Taken together, these data suggest effectiveness of IL-4 in providing relief in DED.

The disclosed IL-4 eye drops increased the number of goblet cells, reduced the presence of T cells and macrophages on the ocular tissue, and improved ocular surface integrity in DED. Thus, IL-4 represents a single cytokine capable of concurrently targeting a spectrum of cells associated with the ocular surface to restore homeostasis in DED. Improvements in ocular disease treatment using a multi-targeted approach facilitated by a single cytokine makes the disclosed composition and method superior to those currently available for DED patients.

### Example 2

### IL-4 Effect of Dermatan Sulfate Complexation on Macrophage Polarization

M1-pro-inflammatory macrophages have an elevated presence in the ocular surface in patients suffering from dye eyes and play a role in perpetuating the pro-inflammatory environment reminiscent of DED. Therefore, modulating the ocular environment from a M1-pro-inflammatory state to a M2-antiinflammatory state is critical in restoring the homeostatic immune environment of the eye. This example describes the effect of IL-4 treatment on polarization of macrophages in the M2-like phenotype and the effect of dermatan sulfate complexation to this process.

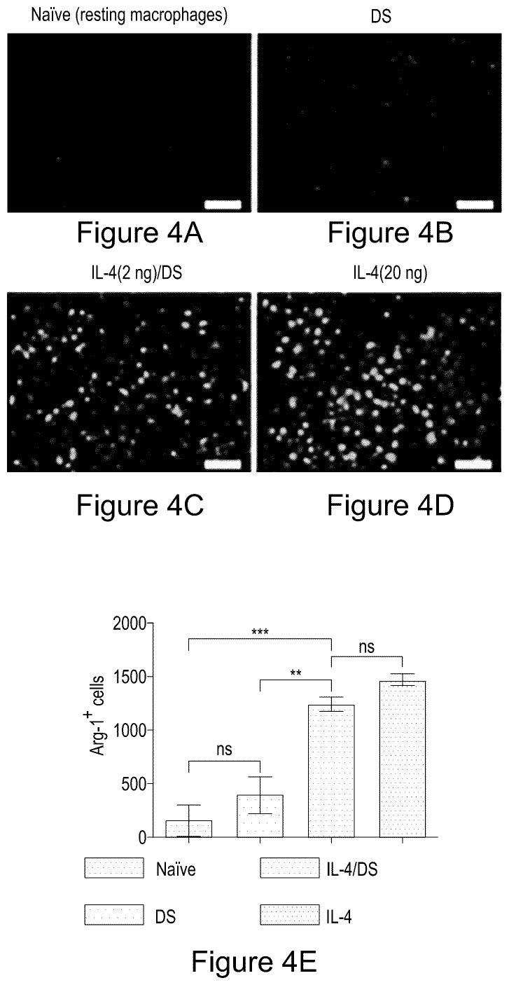

Mouse macrophages were treated with either 20 ng IL-4 or, 2 ng IL-4 complexed with dermatan sulfate (DS). FIGS. 4A-4E show immunostaining and quantitative analysis for expression of the M2 polarization marker arginase-1 (Arg-1). The data shows that complexation with DS significantly reduced the amount of IL-4 needed to elicit transformation of macrophages to a M2-like phenotype (FIG. 4E). Thus, formulations comprising DS-complexed IL-4 provide additional benefits over non-complexed IL-4 formulations by significantly reducing the amount of IL-4 required to produce the same effect.

Thus, the use of IL-4 containing formulations not only favorably modulate both innate and adaptive immune systems but also directly improves tear film stability by restoring goblet cell number and function. The disclosed subject matter provides increased efficacy over current approaches, which rely on the use of products that seek symptomatic relief (e.g., artificial tears products currently in the market), or products that broadly inhibit T cell activation (e.g., cyclosporine, lifitegrast). Accordingly, the disclosed subject matter can be used as an immune cell modulator and a goblet cell fate modulator. Thus, the disclosed subject matter is thus a short term, low-dose, single cytokine-based therapy for DED that not only favorably modulates innate and adaptive immunity, but concurrently improves tear film stability by restoring goblet cell number and function.

### Example 3

### Eye Drop Formulations Comprising IL-4R Signaling Activators in Treatment of Dry Eye Disease.

This example describes utility of an eye drop formulations comprising an activator of IL-4R signaling for treatment of an ocular disease.

Formulation 1—Lyophilized rabbit IL-4 is reconstituted in sterile PBS containing bovine serum albumin (1% w/v) to obtain an IL-4 concentration of 1 ng/30 μL (1 drop).

Formulation 2—Lyophilized rabbit IL-4 is reconstituted in sterile PBS containing dermatan sulfate (2 mg/ml DS) and bovine serum albumin (1% w/v) to obtain an IL-4 concentration of 1 ng/30 μL (1 drop).

Formulation 3—Lyophilized rabbit IL-13 is reconstituted in sterile PBS containing bovine serum albumin (1% w/v) to an IL-13 concentration of 1 ng/30 μL (1 drop).

Formulation 4—Lyophilized rabbit IL-13 is reconstituted in sterile PBS containing dermatan sulfate (2 mg/ml DS) and bovine serum albumin (1% w/v) to obtain an IL-13 concentration of 1 ng/30 μL (1 drop).

Formulation 5—Lyophilized rabbit IL-4 and IL-13 are reconstituted in sterile PBS containing bovine serum albumin (1% w/v) to obtain concentration of 1 ng/30 μL for each of IL-4 and IL-13 (1 drop).

Formulation 6—Lyophilized rabbit IL-4 and IL-13 are reconstituted in sterile PBS containing dermatan sulfate (2 mg/ml DS) and bovine serum albumin (1% w/v) to obtain concentration of 1 ng/30 μL for each of IL-4 and IL-13 (1 drop).

A DED rabbit model is used to test the efficacy of the eye drop formulations. The inferior lacrimal gland and the nictitating membrane are removed from rabbits and DED allowed to develop for 4 weeks (confirmed by fluorescein staining). To test the efficacy of the formulations, DED animals are divided into 7 groups. Groups 1-6 (treatment group) receive formulations 1-6 respectively, administered once daily for 14 days. Group 5 receives PBS and serves as the control group. At 14 days, the status of DED is determined by fluorescein staining. To confirm the status of DED at the cellular level, animals are sacrificed, and histological analysis performed on the resected ocular tissues. The number of goblet cells, RAM11 positive macrophages, and CD3⁺ T cells in the treatment group is assessed by immunostaining and compared with the control group. As compared to the control PBS group, a significantly reduced macrophage and T cell numbers, and an increase in the number of goblet cells is observed for the treatment group.

### BIBLIOGRAPHY

1. Gayton J L (2009) Etiology, prevalence, and treatment of dry eye disease. Clinical Ophthalmology (Auckland, N. Z.) 3:405-412.
2. Kawashima M, et al. (2016) The association of sleep quality with dry eye disease: the Osaka study. Clin Ophthalmol 10:1015-1021.
3. Karakus S, et al. (2018) Impact of Dry Eye on Prolonged Reading. Optometry and vision science: official publication of the American Academy of Optometry 95(12): 1105-1113.
4. Li M, Gong L, Sun X, & Chapin W J (2011) Anxiety and depression in patients with dry eye syndrome. Current eye research 36(1):1-7.
5. Yu J, Asche C V, & Fairchild C J (2011) The economic burden of dry eye disease in the United States: a decision tree analysis. Cornea 30(4):379-387.
6. Dartt D A & Willcox M D P (2013) Complexity of the tear film: importance in homeostasis and dysfunction during disease. Exp Eye Res 117:1-3.
7. Wei Y & Asbell P A (2014) The core mechanism of dry eye disease is inflammation. Eye & contact lens 40(4):248-256.
8. Messmer E M (2015) The pathophysiology, diagnosis, and treatment of dry eye disease. Dtsch Arztebl Int 112(5):71-81; quiz 82.
9. Haber S L, et al. (2019) Lifitegrast: a novel drug for patients with dry eye disease. Ther Adv Ophthalmol 11:2515841419870366-2515841419870366.
10. Periman L M, Perez V L, Saban D R, Lin M C, & Neri P (2020) The Immunological Basis of Dry Eye Disease and Current Topical Treatment Options. Journal of ocular pharmacology and therapeutics: the official journal of the Association for Ocular Pharmacology and Therapeutics 36(3):137-146.
11. Choy B N K, et al. (2019) Factors Associated with Poor Eye Drop Administration Technique and the Role of Patient Education among Hong Kong Elderly Population. Journal of ophthalmology 2019:5962065.
12. Rouen P A & White M L (2018) Dry Eye Disease: Prevalence, Assessment, and Management. Home healthcare now 36(2):74-83.
13. Thode A R & Latkany R A (2015) Current and Emerging Therapeutic Strategies for the Treatment of Meibomian Gland Dysfunction (MGD). Drugs 75(11):1177-1185.
14. Best A L, et al. (2019) [Punctal and canalicular plugs: Indications, efficacy and safety (French translation of the article)]. Journal francais d'ophtalmologie 42(4): 404-414.
15. Shoari A, Kanavi M R, & Rasaee M J (2021) Inhibition of matrix metalloproteinase-9 for the treatment of dry eye syndrome; a review study. Exp Eye Res 205:108523.
16. Liu R, et al. (2017) Analysis of Cytokine Levels in Tears and Clinical Correlations After Intense Pulsed Light Treating Meibomian Gland Dysfunction. American journal of ophthalmology 183:81-90.
17. Na K S, Mok J W, Kim J Y, Rho C R, & Joo C K (2012) Correlations between tear cytokines, chemokines, and soluble receptors and clinical severity of dry eye disease. Invest Ophthalmol Vis Sci 53 (9):5443-5450.
18. Egholm C, Heeb L E M, Impellizzieri D, & Boyman O (2019) The Regulatory Effects of Interleukin-4 Receptor Signaling on Neutrophils in Type 2 Immune Responses. Front Immunol 10:2507.
19. Brown B N, Ratner B D, Goodman S B, Amar S, & Badylak S F (2012) Macrophage polarization: an opportunity for improved outcomes in biomaterials and regenerative medicine. Biomaterials 33(15):3792-3802.
20. Noben-Trauth N, Hu-Li J, & Paul W E (2000) Conventional, naive CD4+ T cells provide an initial source of IL-4 during Th2 differentiation. J Immunol 165(7): 3620-3625.
21. Reyes J L, et al. (2018) Innate and Adaptive Cell Populations Driving Inflammation in Dry Eye Disease. Mediators Inflamm 2018:2532314.
22. Gipson I K (2016) Goblet cells of the conjunctiva: A review of recent findings. Prog Retin Eye Res 54:49-63.
23. Alam J, de Paiva C S, & Pflugfelder S C (2020) Immune—Goblet cell interaction in the conjunctiva. The ocular surface 18(2):326-334.
24. Garcia-Posadas L, Hodges R R, Diebold Y, & Dartt D A (2018) Context-Dependent Regulation of Conjunctival Goblet Cell Function by Allergic Mediators. Sci Rep 8(1): 12162.
25. Fujishima H, Takeuchi T, Shinozaki N, Saito I, & Tsubota K (1995) Measurement of IL-4 in tears of patients with seasonal allergic conjunctivitis and vernal keratoconjunctivitis. Clinical and experimental immunology 102(2):395-398.
26. Griffiths C, et al. (2021) Dupilumab in Adults with Moderate-to-Severe Atopic Dermatitis and Prior Use of Systemic Non-Steroidal Immunosuppressants: Analysis of Four Phase 3 Trials. Dermatol Ther (Heidelb) 11(4):1357-1372.
27. Hachim D, LoPresti S T, Yates C C, & Brown B N (2017) Shifts in macrophagephenotype at the biomaterial interface via IL-4 eluting coatings are associated with improved implant integration. Biomaterials 112:95-107.
28. Lis G J, Litwin J A, Furgal-Borzych A, Zarzecka J, & Cichocki T (2007) Macrophage-specific RAM11 monoclonal antibody cross-reacts with basal cells of stratified squamous epithelia. Folia histochemica et cytobiologica 45(3):229-232.
29. Lis G J, et al. (2010) Expression of basal cell marker revealed by RAM11 antibody during epithelial regeneration in rabbits. Folia histochemica et cytobiologica 48 (1): 89-92.
30. Farkouh A, Frigo P, & Czejka M (2016) Systemic side effects of eye drops: a pharmacokinetic perspective. Clinical ophthalmology 10: 2433-2441.
31. Mun Y, Hwang J S, & Shin Y J (2021) Role of Neutrophils on the Ocular Surface. Int J Mol Sci 22.
32. You I C, et al. (2015) Macrophage Phenotype in Ocular Surface of Experimental Murine Dry Eye Disease. Arch Immunol Ther Exp (Warsz) 63:299-304.
33. Pflugfelder S C, Corrales R M, & de Paiva C S (2013) T helper cytokines in dry eye disease. Exp Eye Res 117:118-125.
34. Reji M A, Haque A, Goyal S, & Krishnaswamy G (2022) Dupilumab-induced ocular surface disease: a primer. BMJ Case Rep 15.
35. Wang Y & Jorizzo J L (2021) Retrospective analysis of adverse events with dupilumab reported to the United States Food and Drug Administration. J Am Acad Dermatol 84: 1010-1014.

All patents, patent applications, publications, product descriptions, and protocols, cited in this specification are hereby incorporated by reference in their entireties. In case of a conflict in terminology, the present disclosure controls.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain using no more than routine experimentation, many equivalents to the specific embodiments described herein. Such equivalents are intended to be encompassed by the following claims.

Various patents and patent applications are cited herein, the contents of which are hereby incorporated by reference herein in their entireties.

What is claimed is:

1. A composition for treating an ocular disorder comprising an effective amount of an interleukin protein, wherein the interleukin protein comprises interleukin-4 (IL-4), interleukin-13 (IL-13), or a combination thereof.

2. The composition of claim 1, further comprising a dermatan sulfate complexed with the interleukin protein.

3. The composition of claim 2, wherein the dermatan sulfate is complexed with the interleukin protein in an interleukin protein:dermatan sulfate ratio from about 1:1000 w/w to about 1:120000 w/w.

4. The composition of claim 3, wherein the interleukin protein:dermatan sulfate ratio is about 1:60000 w/w.

5. The composition of claim 1, further comprising a buffer with a carrier protein.

6. The composition of claim 5, wherein the buffer is phosphate-buffered saline (PBS), and the carrier protein is bovine serum albumin.

7. The composition of claim 1, wherein the ocular disorder comprises dry eye syndrome, conjunctivitis, hematomas, corneal abrasion, or combinations thereof.

8. The composition of claim 1, wherein the interleukin protein is in a concentration range from about 0.1 ng to about 50 ng.

9. The composition of claim 1, wherein the dermatan sulfate is in a concentration range from about 100 ng to about 6000 μg.

10. The composition of claim 1, wherein the composition is in a form of eye drop solution.

11. The composition of claim 10, wherein a concentration of the interleukin protein is from about 1 ng of the interleukin protein per 5 μL of the solution to about 1 ng of the interleukin protein per 500 μL of the solution.

12. The composition of claim 10, wherein each drop of the eye drop solution corresponds to an amount of the interleukin protein from about 0.1 ng to about 50 ng.

13. A method for treating an ocular disorder in a subject comprising administering to the eye of a subject, a composition comprising an effective amount of a composition of claim 1.

14. The method of claim 13, wherein the composition is administered topically to the eye of the subject.

15. The method of claim 14, wherein the composition is administered in a form of an eye drop solution.

16. The method of claim 13, wherein the composition is administered to the eye of the subject in a dose range from about 0.1 ng per eye to about 10 ng per eye.

17. The method of claim 15, wherein the dosage is 2 drops of the eye drop solution, wherein each drop has a volume of about 30 μL.

18. The method of claim 17, wherein the composition is administered to the eye of the subject daily, or twice daily for about 14 days.

19. The method of claim 13, wherein the ocular disorder is dry eye syndrome, conjunctivitis, hematomas, corneal abrasion, or combinations thereof.
